(19) United States
(12) Patent Application Publication
(10) Pub. No.: US 2014/0350580 A1
(43) Pub. Date: Nov. 27, 2014

(54) HERNIA REPAIR DEVICE WITH CORE AND ADVANCED PRE-PERITONEAL DISK DEPLOYMENT

(71) Applicant: Insightra Medical Inc., Irvine, CA (US)

(72) Inventor: Stephen Graham Bell, Rome (IT)

(21) Appl. No.: 14/452,916

(22) Filed: Aug. 6, 2014

Related U.S. Application Data
(63) Continuation-in-part of application No. 13/443,266, filed on Apr. 10, 2012.
Application No. 13/443,266 is a continuation-in-part of application No. 12/183,930, filed on Jul. 31, 2008.
Application No. 12/183,930 is a continuation-in-part of application No. 13/476,202, filed on May 21, 2012.
Application No. 13/476,202 is a continuation-in-part of application No. 12/183,930, filed on Jul. 31, 2008.
Application No. 12/183,930 is a continuation-in-part of application No. 12/361,148, filed on Jan. 28, 2009.
(60) Provisional application No. 61/877,232, filed on Sep. 12, 2013.
(60) Provisional application No. 61/598,254, filed on Feb. 13, 2012.
(60) Provisional application No. 61/013,619, filed on Dec. 13, 2007.
(60) Provisional application No. 61/030,439, filed on Feb. 21, 2008.
(60) Provisional application No. 61/024,489, filed on Jan. 29, 2008.
(60) Provisional application No. 61/097,756, filed on Sep. 17, 2008.

(51) Int. Cl. A61F 2/00

(52) U.S. Cl. CPC A61F 2/0063
USPC 606/151

(57) ABSTRACT

An implant to repair a hole in a muscle wall includes a flat mesh with a central resilient core which deforms to place the core in a hole in a muscle wall and then resiliently expands when released to urge against the perimeter of the hole, dynamically “riding” in the hole as the hole perimeter expands and contracts. Straps on the mesh are tunneled through tissue in the muscle wall containing the hole to hold the mesh with core in place.

Priority is claimed to U.S. provisional application 61/877,232, filed Sep. 12, 2013.

This application is a continuation-in-part of U.S. patent application Ser. No. 12/361,148, filed Jan. 28, 2009, which claims priority to U.S. provisional application 61/024,489, filed Jan. 29, 2008 and to U.S. provisional application 61/097,756, filed Sep. 17, 2008.

The application also is a continuation-in-part of U.S. patent application Ser. No. 13/443,266, filed Apr. 10, 2012, which claims priority to U.S. provisional application 61/598,254, filed Feb. 13, 2012 and which is a continuation-in-part of U.S. patent application Ser. No. 12/183,930, filed Jul. 31, 2008, which in turn claims priority to U.S. provisional applications 61/013,619, filed Dec. 13, 2007 and 61/030,439, filed Feb. 21, 2008. This application is moreover a continuation-in-part of U.S. patent application Ser. No. 13/476,202, filed May 21, 2012, which is a continuation-in-part of the above-referenced U.S. patent application Ser. No. 12/183,930.

Priority to all of the above-referenced applications is claimed. All of the above-referenced applications are incorporated herein by reference. The U.S. patent publication numbers corresponding to the above-referenced utility patent applications are 2009/0192530; 2012/0209301; 2009/0216253; and 2012/0232334, all of which are incorporated herein by reference. Also incorporated by reference is USPP 2008/0287970.

### FIELD OF THE INVENTION

The present application relates generally to the repair of defects in muscular structures, and more particularly to repairing hernias.

### BACKGROUND OF THE INVENTION

A hernia is a condition in which part of the intestine bulges through a weak area in muscles of the abdomen. The main treatment for inguinal hernia is surgery to block the protrusion of abdominal content through the muscle wall.

### SUMMARY OF THE DISCLOSURE

An apparatus includes a flexible mesh including a mesh body and plural flexible straps extending radially away from the body substantially parallel to the body and lying flat on a surface with the mesh when the mesh is placed flat on a surface. At least one plug or 3D structure designed to fill a defect is on the mesh and has plural lobes defining a petal configuration.

The straps can be made integrally with the mesh or may not be made integrally with the mesh. At least one strap defines side edges and laterally-extending elements on at least one side edge. An anti-adhesion substance may be on the mesh. The mesh may be synthetic, biologic, absorbable, semi-absorbable or a mixture of all of these.

In another aspect, a method for treating a defect in a muscle wall includes advancing a flexible mesh adjacent the muscle wall, and locating a resilient core on the mesh in the defect to block the defect. The method also includes tunneling, through tissue, straps extending laterally away from the mesh to locate and/or to hold the mesh against the muscle wall with the resilient core blocking the defect.

In another aspect, an implant to repair a hole in a muscle wall includes a flat mesh and a resilient core centrally located on the mesh. Or the core may be offset from the center of the mesh. The core deforms to place the core in a hole in a muscle wall and then resiliently expands when released to urge against the perimeter of the hole, dynamically “riding” in the hole as the hole perimeter expands and contracts. Plural straps on the mesh are configured to be tunneled through tissue in the muscle wall containing the hole to hold the mesh with core in place. The straps also are designed to facilitate the spreading of the underlay mesh. This can be a difficult maneuver and the strap helps to spread the underlay, in lieu of or in addition to fixing the mesh in the patient. In some embodiments the mesh uses the strap to fix—in others merely to pull and extend the mesh like a ‘winch’. Hence the strap may be detachable or trimmed post placement of the underlay mesh.

The details of the present application, both as to its structure and operation, can best be understood in reference to the accompanying drawings, in which like reference numerals refer to like parts, and in which:

### BRIEF DESCRIPTION OF THE DRAWINGS

FIG. 1 is a perspective view of an example mesh with straps and plug portion; and

FIG. 2 is a schematic view of the plug located in a muscle defect and the mesh straps pulled through the muscle to locate the mesh, showing a strap pulling tool in an exploded relationship to a strap.

### DETAILED DESCRIPTION OF THE PREFERRED EMBODIMENT

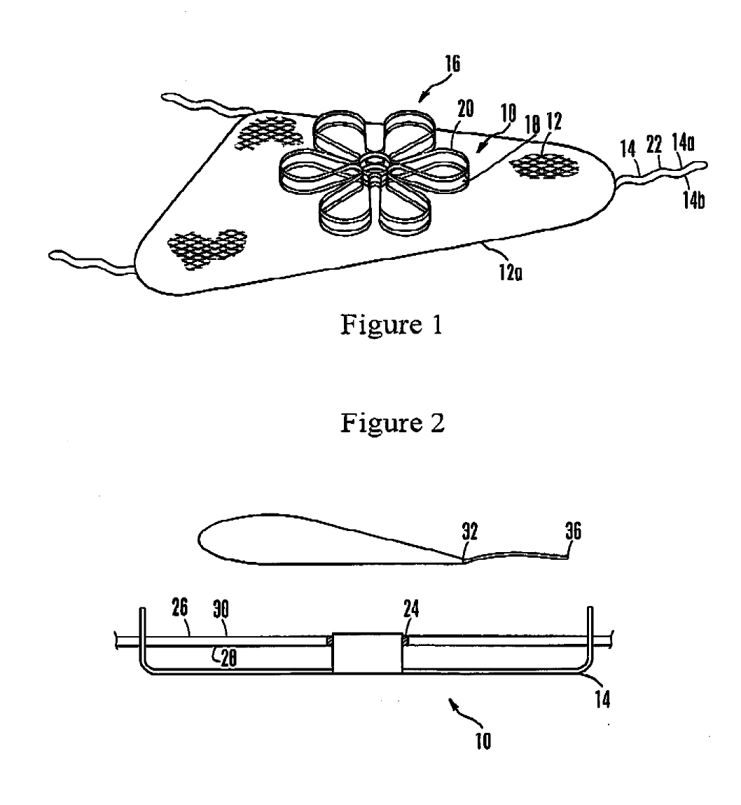

FIG. 1 shows a 10 that includes a mesh 12 made of flaccid, tensionable material and plural straps 14 extending radially away from the mesh. The mesh 12 may be made of synthetic strands or it may be derived from biologic animal material or a combination of both biologic and strands. With more specificity, the term “biologics” refers to meshes or hernia implants that are derived from biologic sources such as pericardium, dermis, serosal layers etc. They are not synthetic and they act through a different tissue integration mechanism than synthetics, namely, biologics act as acellular scaffolds. “Biomimetics” on the other hand are synthetically derived acellular scaffolds, such as the products sold under the trade names REVIVE and ASSURE and GORE BIO A. Regardless, present principles may use all synthetic meshes, all biologic meshes, all biomimetic meshes, or a mixture such as a synthetic core sewn using a suture to a Biologic pre-peritoneal patch. An example way to attach a biologic mesh to either synthetic or a “core” to a “patch” as describe below would be via Prolene sutures.

The straps 14 can also be flaccid so that the strap can lie flat in the same plane as the mesh and be pulled through muscle passageways according to description below to locate and hold the mesh in place. That is, the flexible straps extend radially away from the body substantially parallel to the body and lie flat on a surface with the mesh when the mesh is placed flat on a surface. The mesh 12 with straps 14 may be implemented by any one of the meshes with straps described in the referenced publications.

A resilient deformable plug 16 that may be made of mesh material is located on the mesh 12, preferably centrally located thereon, for filling a defect in a muscle wall. The plug 16 may be established by any of the plugs described in the referenced documents above. The plug 16 may be sewn to the mesh 12, adhered to the mesh 12 using biocompatible adhesive, attached to the mesh 12 using rf sealing or sonic welding, or connected to the mesh 12 using appropriate connection methods for the particular materials selected for the implant.

In the embodiment shown, the plug 16 can be formed of a ribbon of mesh strands in a flower petal configuration as shown, in which plural flexible lobes 18 that may be oblong-shaped or oval-shaped or catenary-shaped. The plug 16 may be provided with strengthening members in accordance with disclosure below. Briefly, in the example shown a strengthening member 20 is provided around the peripheries of the tops of each lobe 18 of the plug 16.

In some embodiments, the strengthening member 20 is established by thread fibers that are more closely knitted together than the fibers of the mesh which make up the plug 16. The fibers of the strengthening member 20 which individually may be the same size or smaller than the fibers of the plug 16 can be woven (including as by knitting or sewing) into the fibers of the plug 16. This creates additional stiffness by concentrating more material in one area, resulting in increased fiber density, increased thickness, or both.

In example non-limiting embodiments the plug 16 and/or mesh 12 can be knitted in an open weave pattern, using polypropylene fibers three to seven mils in diameter. In any case, the plug 16 can exhibit both stiffness and elasticity, so that the combination of structure has a resistance to crush, but can still return to an original configuration if deformed. In some embodiments the overall amount of material may be minimized, and the stiffness can be anisotropic. This may be achieved by increasing the fiber density in specific regions in the same manner as described above.

In greater detail, a knitted mesh material can be knitted into a strip with a more open knit in the middle (pore size of between eight-tenths of a square millimeter and sixteen square millimeters (0.8 mm²-16 mm²) and significantly greater fiber density (length of fiber in a given area) at the edges (fiber density ten to one hundred times greater than in the base material) using a polypropylene fiber three to seven mils in thickness. This strip can then be heat set into a final weave configuration and further heat set into a petal configuration. This particular method creates resistance to circumferential crush on the sides of the petals, but minimal resistance to crush from the top.

As the fiber thickness increases, the stiffness increases, but the elasticity (i.e. the ability to return to a given shape after being deformed) decreases. Therefore, the amount of fibers and fiber thickness can be established to obtain the desired combination of stiffness and elasticity. Specifically, in example non-limiting embodiments the plug 16 can be knitted of a polypropylene fiber of between four to seven mils in diameter while the fibers that establish the strengthening member 20 can be one-half mil to three mils smaller in diameter than those used in the plug 16, and can be knitted to the edges of the plug 16 in a denser configuration to produce specific material properties. To increase the resistance to crush from the top, additional fibers may be knitted in a sinusoidal pattern into the middle of the plug 16.

It is to be appreciate from the above disclosure that the plug 16 establishes a central dynamic core that acts to anchor the implant into a hernia defect or other tissue defect. The disk of flat mesh 12 is attached to the plug 16 and can be placed in the preperitoneal space and also can be placed between any two tissue planes.

The straps 14 can be used to both position the flat mesh and to hold the flat mesh in position next to anatomical tissues. A strap 14 can be of any appropriate length. In one embodiment a strap is long enough to be placed prior to the plug 16 being deployed into the hernia (defect) space. This allows a pulley type movement which “hoists” the flat mesh 12 into the correct position of an anatomical plane.

The mesh 12 can be flat and flexible and can have a periphery 12a of any suitable geometrical shape, e.g., roughly triangular as show, or rectilinear, or circular, or ovular, or racetrack-shaped. Preferably the shape of the periphery fits the anatomical space where it will be deployed.

As shown in FIG. 1, plural straps 14 may be provided to allow the mesh 12 to be laid flat in different directions. One or more straps 14 can be “radiused” to cause a bunching of strap that will act as a natural tissue stop, thus preventing excessive force being applied to the body. For example, as shown in FIG. 14, instead of having side edges 14a, 14b that are linear when the strap 14 is laid flat onto a surface, the side edges 14a, 14b may be formed with scallops or other lateral protrusions 22, including thread strands, etc. Such structure is described in the above-incorporated USPP 2012/0232334.

The straps 14 may be made integrally with the mesh 12 or may be made as a separate component from the mesh 12 and attached to the mesh 12, e.g., by sewing. The straps 14 can be made of permanent material, biologically absorbable or semi-absorbable material, and may be made of synthetic materials or biological materials.

Without limitation the mesh can be made of polypropylene, expanded polytetrafluoroethylene (PTFE), polyester, biodegradable materials, the material marketed as “dualmesh”, a trademark of W.L. Gore, or even metal such as stainless steel or nitinol, or some combination thereof. The mesh may have one or more layers constructed from a bioabsorbable material such that the mesh may be reabsorbed by the body over time. The mesh may have one or more layers constructed from a layer having anti-adhesion properties such that ingrowth or attachment of tissue to the mesh is inhibited. One or more layers may also be coated with an anti-adhesional coating that is applied to a surface to inhibit tissue attachment. These anti-adhesional characteristics may be particularly useful for those implant surfaces that are exposed to the internal viscera of the abdominal cavity. In this situation it may be helpful to inhibit potential attachment of various organs to the implant. This may be particularly possible if the innermost surface of the ventral wall, the peritoneum 18, is compromised. One example of an adhesion resistant material is, for example, a thread of polytetrafluoroethylene polymer material of the type sold under the trade name “Gore-Tex” by W. L. Gore & Associates, Inc. As stated above, the mesh may also be made of biologic material derived from animal tissue.

Referring now to FIG. 2, the device 10 can block an opening 24 of a muscle wall 26 that may have an anterior surface 28 and a posterior surface 30. The device 10 can be moved between an insertion configuration, in which the plug 16 of the device 10 is compressed to be smaller than the opening 24 to facilitate advancing the plug 16 into the opening, and an implanted configuration (FIG. 2), in which the plug 16 is released to resiliently expand to fill the opening 24 and dynamically move as the tissue surrounding the opening moves. In this configuration, mesh 12 is substantially flat and unwrinkled/unfolded and is larger than the opening 24. The plug 16 is biased to the implanted configuration at least by the strengthening member 20. Thus, the plug 16 is resilient and is materially biased to the implanted configuration.

The wall 26 may be, as an example, a wall of an abdomen muscle in which the opening 24 has formed as a hernia, such as the ventral wall. Defects in other muscle walls may be similarly resolved using the device 10. Other muscle wall defects such as pelvic floor prolapse may be similarly resolved.

With particular regard to the application shown in FIG. 2, the mesh straps 14 are tunneled through tissues by means of a needle, penetrating instrument or specific strap passing device 32, which grips a strap 14 and/or tunnels through tissue. Non-limiting examples of such tools are divulged in the above-incorporated USPPs 2012/0209301; 2009/0216253; and 2012/0232334. For deployment, the mesh straps 14 can be disposed in a tissue tunnel which has a smaller diameter than the diameter of the strap, thus allowing a frictional placement. The strap can remain in place in the body by friction, tissue ingrowth, or if desired the straps can be sutured, glued or affixed by various other means to the tissue and/or removed after mesh placement.

A mesh deployment mechanism can be used in conjunction with the dynamic core or for flat meshes in general. Manipulation of the needle of such a mechanism, such as the needle 36 of the tool 36 shown in FIG. 2, can be performed as an outside in or inside out pass from the anatomical space where the mesh 12 will lie. Or, an integral needle/tunneler can be swaged onto the end of the mesh strap much like a suture.

An example non-limiting embodiment may include the above-described core as described in the referenced applications sewn onto a biologic pericardium patch (to establish the “mesh”) which in turn has one or more synthetic polypropylene arms sewn to it using polypropylene suture. A second embodiment may include a plug of Biologic material made from a rolled up piece of biologic sewn to a polypropylene patch (which establishes the “mesh”) that has integrated polypropylene arms for fixation.

While the particular HERNIA REPAIR DEVICE WITH CORE AND ADVANCED PRE-PERITONEAL DISK DEPLOYMENT is herein shown and described in detail, it is to be understood that the subject matter which is encompassed by the present invention is limited only by the claims.

What is claimed is:

1. Apparatus, comprising:
a flexible mesh including a mesh body and plural flexible straps extending radially away from the body substantially parallel to the body and lying flat on a surface with the mesh when the mesh is placed flat on a surface; and
at least one plug on the mesh with plural lobes defining a petal configuration.

2. The apparatus of claim 1, wherein the straps are made integrally with the mesh.

3. The apparatus of claim 1, wherein the straps are not made integrally with the mesh.

4. The apparatus of claim 1, wherein at least one strap defines side edges and laterally-extending elements on at least one side edge.

5. The apparatus of claim 1, comprising an anti-adhesion substance on the mesh.

6. Method for treating a defect in a muscle wall, comprising:
advancing a flexible mesh adjacent the muscle wall;
locating a resilient core on the mesh in the defect to block the defect; and
tunneling straps extending laterally away from the mesh through tissue to locate and/or hold the mesh against the muscle wall with the resilient core blocking the defect.

7. The method of claim 6, wherein the defect is an inguinal hernia.

8. The method of claim 6, comprising using a needle to execute the tunneling.

9. The method of claim 6, comprising leaving the straps in tissue without fastening the straps to tissue to hold the mesh in place without sutures or staples and then closing a patient in whom the defect is treated.

10. The method of claim 6, comprising fastening the straps to tissue.

11. The method of claim 6, comprising using a resilient core with a lobed shape to block the defect.

12. An implant to repair a hole in a muscle wall, comprising:
a flat mesh;
a resilient core centrally located on the mesh which deforms to place the core in a hole in a muscle wall and then resiliently expands when released to urge against the perimeter of the hole, dynamically “riding” in the hole as the hole perimeter expands and contracts; and
plural straps on the mesh configured to be tunneled through tissue in the muscle wall containing the hole to hold the mesh with core in place.

13. The implant of claim 12, wherein the core includes plural lobes defining a petal configuration.

14. The implant of claim 12, wherein the straps are made integrally with the mesh.

15. The implant of claim 12, wherein the straps are not made integrally with the mesh.

16. The implant of claim 12, wherein at least one strap defines side edges and laterally-extending elements on at least one side edge.

17. The implant of claim 12, comprising an anti-adhesion substance on the mesh.
